(19) United States
(12) Patent Application Publication
(10) Pub. No.: US 2006/0067911 A1
(43) Pub. Date: Mar. 30, 2006

(54) Metered medication dose

(75) Inventors: Thomas Nilsson, Mariefred (SE); Claes Friberg, Styckebruk (SE); Lars Kax, Nykvarn (SE); Alf Niemi, Strangnas (SE); Mattias Myrman, Stockholm (SE); Sven Calander, Strangnas (SE)

Correspondence Address: OBLON, SPIVAK, MCCLELLAND, MAIER & NEUSTADT, P.C., 1940 DUKE STREET, ALEXANDRIA, VA, 22314, US

(73) Assignee: MEDERIO AG, Hergiswil NW (CH)

(21) Appl. No.: 11/111,888

(22) Filed: Apr. 22, 2005

(30) Foreign Application Priority Data
Sep. 24, 2004 (SE) SE 0402345-3

(51) Int. Cl. A61K 38/28; A61K 38/22; A61K 38/19

(52) U.S. Cl. 424/85.1; 514/3; 514/12

(57) ABSTRACT

The invention discloses a metered medication dose of a dry powder protein medicament, particularly a peptide medicament, intended for inhalation by use of an adapted dry powder inhaler. An active peptide agent is presented in a pure, natural, crystalline, micronized, dry powder form. A dose comprises at least one such peptide powder and may optionally comprise at least one biologically acceptable excipient in dry powder form. The dose does not include any substances that are intended to change one, some or all properties of the at least one peptide with an object of improving the stability or systemic absorption of the active peptide or peptides deposited.

### REFERENCE TO PRIOR APPLICATIONS

This application claims the benefit of SE 0402345-3 filed Sep. 24, 2004 which is incorporated herein by reference.

### TECHNICAL FIELD

The present invention relates to a metered medication dose of a peptide medicament in dry powder form adapted for a dry powder inhaler, more particularly to a dose comprising at least one finely divided, systemically acting, pure peptide dosage for deep lung deposition and systemic delivery.

### BACKGROUND

Supplying medication drugs directly to the airways and lungs of a patient by means of an inhaler is an effective, quick and user-friendly method of drug delivery. Because the efficacy of inhaled doses often are much higher than e.g. orally administered capsules or pills, the inhalation doses need only be a fraction of the medicament mass in an oral dose. A number of different devices have been developed in order to deliver drugs to the lung, e.g. pressurized aerosol inhalers (pMDIs), nebulizers and dry powder inhalers (DPIs).

While inhalation of drugs already is well established for local treatment of respiratory diseases such as asthma, much research is going on to utilize the lung as a feasible entry into the body of systemically acting drugs. For locally acting drugs, the preferred deposition of the drug in the lung depends on the localization of the particular disorder, so depositions in the upper as well as the lower airways are of interest. For systemic delivery of the medication, a deep lung deposition of the drug is preferred and usually necessary for maximum efficiency. The expression “deep lung” should be understood to mean the peripheral lung and alveoli, where direct transport of active substance to the blood can take place.

The lung is an appealing site for systemic delivery of drugs as it offers a large surface area (about 100 m²) for the absorption of the molecules across a thin epithelium, thus having a potential for rapid drug absorption. Pulmonary delivery of drugs has the potential of attaining a high, rapid systemic drug concentration without the need of enhancers. The feasibility of this route of administration for a particular drug depends on, for example, dose size and extent of systemic absorption of the particular drug. The critical factors for the deposition of inhaled particles in the lung are inspiration/expiration pattern and the particle aerodynamic size distribution. The aerodynamic particle size of the drug particles is important if an acceptable deposition of the drug within the lung is to be obtained. If a particle is to reach into the deep lung the aerodynamic particle size should typically be less than 3 μm, and for a local lung deposition, typically about 5 μm. Larger particle sizes will easily stick in the mouth and throat. Thus, it is important to keep the aerodynamic particle size distribution of the dose within tight limits to ensure that a high percentage of the dose is actually deposited where it will be most effective.

De-Aggregation

Powders with a particle size suitable for inhalation have a tendency of aggregating, in other words to form smaller or larger aggregates, which then have to be de-aggregated before the particles enter into the airways of the user. De-aggregation is defined as breaking up aggregated powder by introducing energy e.g. electrical, mechanical, pneumatic or aerodynamic energy. The aerodynamic diameter of a particle of any shape is defined as the diameter of a spherical particle having a density of 1 g/cm³ that has the same inertial properties in air as the particle of interest. If primary particles form aggregates, the aggregates will aerodynamically behave like one big particle in air.

Most finely divided powders are prone to forming particle aggregates. This tendency is aggravated in the presence of water and some powders are sensitive to very small amounts of water. Under the influence of moisture the formed aggregates require very high inputs of energy to break up in order to get the primary particles separated from each other. Another problem afflicting fine medication powders is electro-static charging of particles, which leads to difficulties in handling the powder during dose forming and packaging. A method and a device for de-aggregating a powder is disclosed in our U.S. Pat. No. 6,513,663 B1. Preferably, the de-aggregating system should be as insensitive as possible to the inhalation effort produced by the user, such that the delivered aerodynamic particle size distribution in the inhaled air is largely independent of the inhalation effort. A very high degree of de-aggregation presumes the following necessary steps:
a suitable formulation of the powder (particle size distribution, particle shape, adhesive forces, density, etc)
a suitably formed dose of the powder adapted to the capabilities of a selected inhaler device
an inhaler device providing shear forces of sufficient strength in the dose to de-aggregate the powder (e.g. turbulence)
   Powder Preparation

Turning to the drug formulation, there are a number of well-known techniques to obtain a suitable primary particle size distribution to ensure correct lung deposition for a high percentage of the dose. Such techniques include jet-milling, spray-drying and super-critical crystallization. There are also a number of well-known techniques for modifying the forces between the particles and thereby obtaining a powder with suitable adhesive forces. Such methods include modification of the shape and surface properties of the particles, e.g. porous particles and controlled forming of powder pellets, as well as addition of an inert carrier with a larger average particle size (so called ordered mixture). A simpler method of producing a finely divided powder is milling, which produces crystalline particles, while spray-drying etc produces amorphous particles. Novel drugs, both for local and systemic delivery, often include biological macromolecules, which put completely new demands on the formulation. In our publication WO 02/11803 (U.S. Pat. No. 6,696,090) a method and a process is disclosed of preparing a so called electro-powder, suitable for forming doses by an electro-dynamic method. The disclosure stresses the importance of controlling the electrical properties of a medication powder and points to the problem of moisture in the powder and the need of low relative humidity in the atmosphere during dose forming.

Dose Forming

Methods of dose forming of powder formulations in prior art include conventional mass, gravimetric or volumetric metering and devices and machine equipment well known to the pharmaceutical industry for filling blister packs and gelatin capsules, for example. See WO 03/66437 A1, WO 03/66436 A1, WO 03/26965 A1, WO 02/44669 A1, DE 100 46 127 A1 and WO 97/41031 for examples of prior art in volumetric and/or mass methods and devices for producing metered doses of medicaments in powder form. Electrostatic forming methods may also be used, for example as disclosed in U.S. Pat. No. 6,007,630 and U.S. Pat. No. 5,699,649.

Packaging

A common dose container in prior art is a gelatin capsule. A gelatin capsule contains typically 13-14% water by weight in the dose forming stage and after the capsules have been loaded, they may be dried in a special process in order to minimize water content. A number of filled gelatin capsules, whether dried or not, are often enclosed in a blister package. The remaining quantity of water in the capsule material is then also enclosed in the blister package. The drive towards equilibrium between the captured air inside the package and the gelatin capsule will generate a relative humidity inside the blister package that will negatively affect the fine particle fraction (FPF) of the powder dose, if the powder is at all moisture sensitive. Drugs in fine powder form, including peptides like insulin, agglomerate easily in the presence of moisture, and the agglomerates are then extremely difficult to de-agglomerate even with high input of de-agglomeration energy. Aseptic filling of gelatin capsules is very difficult and complicated, so in case aseptic production is required it is better to choose a different enclosure for the dose.

A blister pack is a better choice of package for moisture sensitive doses, although a blister of aluminum foil or technical polymer or a combination thereof is sometimes difficult to open for dose access. Peelable blister constructions are sometimes used to improve dose accessibility inside a DPI, but at the price of a less efficient moisture barrier.

Proteins and Peptides

A number of proteins, which per definition includes poly-peptide drugs (PPDs), have a potential for being suitable for inhalation therapy and some of them are in various stages of development. Some examples are insulin, alpha1-proteinase inhibitor, interleukin 1, parathyroid hormone, genotropin, colony stimulating factors, glucagons, glucagon-like peptides, dipeptidyl-peptidase-4, erythropoietin, interferons, calcitonin, factor VIII, alpha-1-antitrypsin, follicle stimulating hormones, LHRH agonist and IGF-1. PPDs have characteristics that present significant formulation challenges. In particular their chemical and enzymatic lability practically prevents traditional dosage forms such as oral tablets. Fortunately, proteins and peptides of moderate molecular weights are soluble in the fluid layer in the deep lung and dissolve, therefore ensuring rapid absorption from the lung. From a stability point of view, a solid formulation stored under dry conditions is normally the best choice. In the solid state, these molecules are normally relatively stable in the absence of moisture or elevated temperatures. For example, insulin in dry powder form is relatively sensitive to moisture, more or less so depending on the formulation and needs to be well protected from moisture up to the point of administration in order to preserve the FPF of the metered dose, which secures a high and stable delivered fine particle dose (FPD).

In the absence of appropriate, inhalable, dry powder doses and suitable DPIs, poly-peptide drugs are currently mainly administered parenterally as intravenous, intramuscular or subcutaneous injections. While these routes are normally satisfactory for a limited number of administrations, there are problems with a long-term therapy. Frequent injections, necessary for the management of a disease, is of course not an ideal method of drug delivery and often leads to a low patient compliance as they infringe on the freedom of the patient and because of psychologic factors in the patient.

Insulin

Insulin is an example of an important peptide drug where frequent parenteral administrations are the most common way of administration. Self-administration of insulin is an important reality and part of everyday life for many patients with diabetes. Normally, the patient needs to administer insulin several times daily. The most common method of insulin administration is subcutaneous injection by the patient based on close monitoring of the glucose level. There are pharmacokinetical limitations when using the subcutaneous route. Absorption of insulin after a subcutaneous injection is rather slow. It sometimes takes up to an hour before the glucose level in the blood begins to be significantly reduced. This inherent problem with subcutaneous insulin delivery cannot be solved with a more frequent administration. To obtain plasma insulin concentrations that are physiologically correct it is necessary to choose another route of administration.

Methods of manufacturing dry powder insulin from a liquid state has been known and applied for more than 50 years, including such methods as evaporation, spray-drying and freeze-drying. Until recently, reliable and economic technologies have been lacking for on one hand producing insulin powders with suitable properties and on the other hand suitable apparatuses for delivering the powder to the user in a way that ensures an effective systemic delivery. This has prevented the mainstream research from using insulin in dry powder formulations. However, in the early 1990's Bäckström, Dahlbäck, Edman and Johansson (Therapeutic preparation for inhalation WO 95/00127) showed that inhalation of a therapeutic preparation comprising insulin and an absorption enhancer quickly and efficiently leads to insulin being absorbed in the lower respiratory tract. It is evident that the enhancer was necessary, probably because of insufficient de-aggregation of the powder and the use of an inferior dry powder inhaler. During the last decade a number of reports describing the pharmacokinetics and pharmacodynamics of insulin delivered to the lung of humans have been published. In most reported cases, the insulin has been nebulized from an aqueous preparation. However, research into the effect of pulmonary administration of insulin in dry powder form has demonstrated that systemic delivery of dry insulin powder can be accomplished by oral inhalation and that the powder can be rapidly absorbed through the alveolar regions of the lung. For instance, in U.S. Pat. No. 5,997,848 it is demonstrated that systemic delivery of dry insulin powder is achieved by oral inhalation and that the powder can be rapidly absorbed through the alveolar regions of the lungs. However, dose resolution still seems to be low. According to the disclosure, the insulin dosages have a total weight from a lowest value of 0.5 mg up to 10-15 mg of insulin and the insulin is present in the individual particles at from only 5% up to 99% by weight with an average size of the particles below 10 μm.

In general, human insulin in dry powder form is presented in modified chemical and/or physical form, such as insulin analogues and/or insulin derivatives, e.g. in order to offer a suitable stability, bioavailability or flowability. Researchers have tested a rather large number of enhancers, and suggested mechanisms are that they open the tight junctions, disrupt membranes or inhibit enzymes. However, when used in nasal inhalation applications, penetration enhancers are known to cause local irritation on the nasal membrane and they may cause detrimental long-term effects in the lung, problems that may prove difficult to solve.

Dry Powder Inhalers

A large number of different concepts to de-aggregate the drug powder in DPIs have been developed. One example is an inhaler coupled to a spacer, a container of relatively large volume for injected aerosolized particles, from which the inhalation can take place. Upon inhalation from the spacer the aerosolized powder will effectively reach the alveoli. This method in principle has two drawbacks, firstly difficulties to control the amount of medicine emitted to the lung, since an uncontrolled amount of powder sticks to the walls of the spacer and secondly difficulties by users in handling the relatively space demanding apparatus.

External sources of energy to amplify the inhalation energy provided by the user during the act of inhalation are common in prior art inhalers for improving the performance in terms of de-aggregation. Some manufacturers utilize electrically driven propellers, piezo-vibrators and/or mechanical vibration to de-aggregate the agglomerates. The addition of external sources of energy leads to more complex and expensive inhalers than necessary, besides increasing the demands put on the user in maintaining the inhaler. An inhaler dosing device is disclosed in our U.S. Pat. No. 6,622,723 B1. A continuous dry powder inhaler is further disclosed in our U.S. Pat. No. 6,422,236 B1. In our publication WO 03/086515 A1 (US 2003/0192538) a device is disclosed setting a new standard for effective aerosolization and delivery of a powder dose. In publication WO 03/086516 A1 (US 2003/0192540) the device is used in a new type of DPI and applied to a metered dose, e.g. insulin, to deliver the fine particle dose to a user of the inhaler, only relying on the power of the inhalation effort.

Hence, there is a demand for suitable therapeutic doses of pure peptide medicaments, without additional substances such as enhancers, and devices for protecting the fine particle dose against moisture from the point of dose manufacture until the dose is administered to the system by inhalation.

### SUMMARY

The present invention discloses a metered medication dose of a dry powder protein medicament, particularly a peptide medicament, intended for inhalation by use of an adapted dry powder inhaler. An active peptide agent, according to the invention, is presented in a pure, micronized, dry powder form. The dose comprises at least one such peptide powder and may optionally comprise at least one biologically acceptable excipient in dry powder form acting as carrier and/or diluent. The dose does not include any substances that are intended to change one, some or all properties of the at least one peptide with an object of e.g. improving the stability or systemic absorption of the active peptide or peptides deposited in the deep lung following an inhalation.

It is an object of the present invention to present a metered dose of at least one peptide medicament, where the fine particle fraction (FPF) of the included peptide or peptides powder(s) is at least 80% by mass, preferably more than 90% by mass, such that the fine particle dose mass (FPD) of the at least one peptide powder, leaving an adapted inhaler, aerosolized into inspiration air is at least 40%, and typically at least 70% of the peptide mass in the metered dose.

The invention teaches that the at least one pharmacologically active peptide agent of the dose is selected from a group comprising rapid, intermediate and slow acting insulin, including insulin analogues, C-peptide of insulin, alpha1-proteinase inhibitor, glucagons, glucagon-like peptides, dipeptidyl-peptidase-4, interleukin 1, parathyroid hormone, genotropin, colony stimulating factors, erythropoietin, interferons, calcitonin, factor VIII, alpha-1-antitrypsin, follicle stimulating hormones, LHRH agonist and IGF-1.

The invention further teaches that the at least one, optional dry excipient comprises an excipient selected from a group consisting of monosaccarides, disaccarides, polylactides, oligo- and polysaccarides, polyalcohols, polymers, salts or mixtures thereof.

According to the disclosure a total metered dose mass is in a range from 0.1 to 50 mg and preferably from 0.5 to 25 mg.

In a further aspect of the present invention a particular peptide powder included in the metered dose is recombinant human insulin powder. The metered dose is adjusted for administration by inhalation.

The present invention also discloses a medical product comprising a metered dose of the protein and preferably peptide medicament in finely divided dry powder form, and a dry, moisture-tight, high barrier seal container, which fits into an adapted dry powder inhaler. The dose loaded into the container, is intended for inhalation and comprises at least one micronized, peptide powder and optionally at least one biologically acceptable excipient powder and does not include any substances that are intended to change one, some or all properties of the at least one peptide with an object of e.g. improving the stability or systemic absorption of the active peptide or peptides. The fine particle fraction (FPF), i.e. the mass of the at least one peptide powder having particles in a range from 1 μm to 5 μm, is kept intact in an amount of more than 80% by mass, preferably more than 90% by mass, by the high barrier seal container for the duration of a shelf life period for the medical product, until the time of administration.

In another aspect of the present invention a particular peptide powder included in the metered dose of the medical product is recombinant, human insulin powder. The medical product is adapted for administration by inhalation.

### BRIEF DESCRIPTION OF THE DRAWINGS

The invention, together with further objects and advantages thereof, may best be understood by referring to the following detailed description taken together with the accompanying drawings, in which:

FIG. 1 illustrates a close-up of two typical, volumetric doses having intact bodies of joined particles and loaded onto a dose bed according to the present invention, and

FIG. 2 illustrates a close-up of a typical, electro-dynamically formed, oblong dose loaded onto a dose bed according to the present invention, and

FIG. 3 illustrates in top and side views a first embodiment of a dose loaded onto a dose bed constituting a part of a high barrier seal container, and

FIG. 4 illustrates in top and side views a second embodiment of a dose loaded onto a dose bed constituting a part of a high barrier seal container.

### DESCRIPTION OF THE INVENTION

The present invention discloses a metered medication dose of a protein medicament and preferably a peptide medicament in dry powder form comprising at least one finely divided active peptide agent optionally in a mixture with at least one biologically acceptable excipient. The dose is intended for inhalation by the use of a dry powder inhaler device. The peptide agent or agents included in the dose are preferably in a pure form without any added substances intended for changing or enhancing one, some or all properties of the peptide(s). The objective of the present invention is to deliver a pure peptide powder dose to the system of a user via the deep lung. No substance besides the active peptide or peptides is included in the dose, except for said optional at least one excipient, which acts as a carrier and/or diluent, but without influencing e.g. the absorption of the peptide. Not having any substance included in the dose, e.g. for enhancing or speeding up peptide absorption through the alveols, for stabilizing the peptide or increasing the bioavailability of the peptide, has the advantage that no such substance can accumulate in the lung or be delivered to the system. The potential threat to the health of the user, especially if a drug is administered on a regular basis, is therefore much less.

However, the quality of a delivered pure peptide dose needs to be very high in terms of fine particle fraction, when no “performance raising” substances are included in the dose. As has been pointed out in the foregoing, particles need to be 5 μm or less in aerodynamic diameter to have a reasonable chance of reaching into the deep lung when inhaled and avoid sticking on the way through the airways to the lung. In the deep lung the small particles may be absorbed by the alveols and delivered to the system. The aerodynamic diameter of particles should preferably be in a range from 0.5 to 5 μm and more preferably in a range 1 to 3 μm for a rapid and successful delivery to the system through the lung. Particles of these sizes sediment in the lung provided that the inhalation is deep and not too short. For maximum lung deposition, the inspiration must take place in a calm manner to decrease air speed and thereby reduce deposition by impaction in the upper respiratory tracts. Particles of aerodynamic diameter less than 1 μm take longer to sediment and a high percentage may not sediment in the lung but follow the expiration air out instead. Small particles are more easily absorbed by the alveols, which is a further reason for the delivered dose, according to the disclosure, to present as high fine particle fraction (FPF) as possible.

The fine particle fraction (FPF) of the finely divided active peptide agent in the metered medicament dose is to be as high as possible, having a mass median aerodynamic diameter (MMAD) below 3 μm and a particle size distribution preferably having more than 80% and most preferably more than 90% by mass of particles with aerodynamic diameter below 5 μm. After forming a metered dose, it is very important to protect the dose from negative influences, which may otherwise detrimentally affect the peptide FPF. Elevated temperatures have negative effects on dose stability by dramatically increasing the rate of break down of the active peptide agent, but moisture also constitutes a particular risk in this respect. In addition, moisture increases the tendency of powders to form agglomerates, which is an even greater concern, since agglomerates lower the FPF of the powder. So, in order to protect the dose according to the present invention against moisture, it is enclosed in a high barrier seal container, whereby the peptide FPF is protected from the point of manufacture to the point of administering the dose, through the steps of transporting, storing, distributing and consuming. Suitable ambient conditions when filling doses are discussed in the following.

Methods of dose forming of protein and peptide powder formulations, according to the present invention, include conventional mass, gravimetric or volumetric metering and devices and machine equipment well known to the pharmaceutical industry for filling blister packs, for example. Electrostatic forming methods may also be used, or combinations of methods mentioned.

A most suitable method of depositing microgram and milligram quantities of dry powders uses electric field technology (ELFID) as disclosed in our U.S. Pat. No. 6,592,930 B2, which is hereby incorporated in this document in its entirety as a reference. In this method powder flowability is unimportant, because powder particles are transported from a bulk source to a dose bed in a dose-forming step, not relying on the force of gravity but using primarily electric and electrostatic force technology to deposit a metered quantity of powder, i.e. a dose, onto the dose bed, which may be a blister, capsule or high barrier container as disclosed in the present invention. An advantage of this electric field dose forming process is that it is not necessary to add large excipient particles to the medicament powder, because good powder flowability is not an issue. Besides optionally contributing desired electrical qualities to the powder, excipients are added, if necessary, to the active peptide agent in order to dilute the drug to have a pre-metered dose in the inhaler exceeding 100 μg.

Surprisingly, we have found by experimentation that the delivered fine particle dose, FPD, of the disclosed metered peptide dose is strongly dependent on the timing of the delivery within the inhalation cycle. Ideally, delivery should begin fairly early in the inhalation cycle, but not until the suction provided by the user has exceeded approximately 2 kPa. Concentrating the suction energy to areas near the metered dose in an adapted DPI may provide a local airflow speed, which is adequate for complete aerosolization and de-aggregation of the dose, particularly if the release of the dose is prolonged, i.e. the dose is arranged to be released gradually and not all at once. The peptide dose is preferably adapted for prolonged delivery within a time frame of not less than 0.1 second and not more than 5 seconds, preferably in a range 0.2-2 seconds. An early delivery of the dose in the inhalation cycle is advantageous, because the aerosolized dose will follow the inspiration air into the empty deep lung and will have time to sediment there. An example of a suitable inhaler is disclosed in our U.S. Pat. No. 6,422,236 B1 and principles of inhaler design are disclosed in our U.S. Pat. No. 6,571,793 B1.

The present invention may advantageously be applied to peptides, such as rapid, intermediate and slow acting insulin including insulin analogues, C-peptide of insulin, alpha1-proteinase inhibitor, glucagons, glucagon-like peptides, dipeptidyl-peptidase-4, interleukin 1, parathyroid hormone, genotropin, colony stimulating factors, erythropoietin, interferons, calcitonin, factor VIII, alpha-1-antitrypsin, follicle stimulating hormones, LHRH agonist and IGF-1.

In a particular embodiment of the present invention the medication dose may comprise a dry powder formulation of a human parathyroid hormone (PTH) for treatment of osteoporosis. PTH therapy results in new bone formation and mineralization occurs not only in the existing protein matrix but also in the new bone structure that is formed. Bone density increases and as a consequence of the PTH treatment the risk of new fractures in persons with osteoporosis decreases dramatically. Administration of PTH is today mainly by way of subcutaneous injection, but the peptide may be advantageously formulated as a micronized dry powder and is most suitable for inhalation and pulmonary systemic delivery.

In another embodiment of the present invention the medication dose may comprise a dry powder formulation of a glucagon-like peptide-1 (GLP-1). GLP-1 is synthesized in intestinal endocrine cells in two principal major molecular forms, as GLP-1(7-36) amide and GLP-1(7-37). These molecules are secreted in response to nutrient ingestion and play multiple roles in metabolic homeostasis following nutrient absorption. Biological activities include stimulation of glucose-dependent insulin secretion and insulin biosynthesis, inhibition of glucagon secretion and gastric emptying and inhibition of food intake. The substance plays an important role in lowering blood glucose levels in diabetics by stimulating the beta-cells in pancreas to produce insulin. A very interesting effect of GLP-1 is that it normalizes blood glucose levels in response to hyperglycemic conditions without the risk of ending up in a hypoglycemic condition. Also, GLP-1 helps control satiety and food intake. The substance therefore constitutes an interesting pharmacological drug, particularly so for treatment of diabetes, preferably in combination with insulin or even as an alternative to a regimen of insulin. See European Patent EP 0 762 890 B1.

GLP-1 is a relatively small peptide molecule with a great potential for inhalation therapy. Fortunately, provided that the GLP-1 powder formulation is constituted of particles of the right size to sediment in the deep lung after inhalation, GLP-1 has been shown to be soluble in the fluid layer in the deep lung and dissolve, thereby ensuring rapid absorption from the lung into the system before enzymatic inactivation sets in. See for instance U.S. Pat. No. 6,720,407.

Yet another particular embodiment of the present invention comprises a dry powder formulation of dipeptidyl-peptidase-4 (DPP-4) inhibitor, such as PHX1149 from Phenomix or NVP-DPP728 from Novartis. Inhibitors of DPP-4 have been shown to stop or diminish rapid degradation of GLP-1 by the DPP-4 enzyme. Inhibiting DPP-4 helps the body to activate normal physiological reponses to food intake by indirectly stimulate insulin secretion, slow digestion, suppress glucose production and decrease appetite. By improving the physiologic response to glucose, DPP-4 inhibitors may prevent or delay onset of diabetes.

In a further aspect of the present invention combinations of peptide doses may be arranged for the benefit of subjects, where a combined dose of peptides offers therapeutic advantages compared to separate or just single dose delivery. For example, doses of GLP-1 and DPP-4 may be combined for simultaneous or sequential delivery in a singel inhalation, or combinations of insulin and GLP-1 or insulin and DPP-4 or the three peptides combined are equally possible. See our U.S. Application US-2004-0258625.

From a stability point of view, a solid formulation stored under dry conditions is normally the best choice for embodiments of the present invention, including medicament doses containing insulin, PTH, GLP-1 or DPP-4 inhibitors. In the solid state, these molecules are normally relatively stable in the absence of moisture or elevated temperatures. Generally, peptides in dry powder form suitable for inhalation are more or less sensitive to moisture and protecting the metered medication dose from moisture all the way through the steps of filling, sealing, transporting and storing is an important aspect of the present invention.

A particular peptide of the present invention is insulin, insulin analogues and insulin derivatives, preferably recombinant, human insulin. A dry powder of insulin, suitable for use in the present invention, is preferably in crystalline form rather than amorphous form. The limit for water content of the powder is set as low as possible, not exceeding 10% (w/w) and preferably below 5% (w/w). Prior art methods of producing an insulin powder generally involves spray-drying, freeze-drying, vacuum drying or open drying, which methods result in an amorphous powder. Generally, amorphous insulin is less stable than crystalline insulin, which explains why it is common in prior art to include a stabilizing agent, besides other substances for various purposes. A preferred method of preparing a dry, crystalline insulin powder before an optional mixing step, is to mill the insulin powder at least once and preferably twice by jetmilling in order to get a small MMAD for the micronized powder in a range 1-3 μm with as small tails of particles outside this range as possible. In our experience there is no deterioration of the insulin stability because of milling in this way. The micronized powder is then optionally mixed with one or more excipients in order to dilute the potency of the insulin and to get a powder well adapted to chosen methods of metering and forming doses. In another aspect of the present invention it is advantageous to include more than one formulation of recombinant, human insulin powder in the dose, e.g. in order to improve the insulin delivery into the blood circulation, such that the natural course of insulin production in a healthy person is mimicked more closely than would be possible when using only one insulin formulation. Different formulations of recombinant insulin present different absorption delays and blood concentrations over time. Therefore, a combination of two or more insulin analogues in a dose is well suited with the objective of adjusting the systemic concentration of insulin in the blood of a diabetic user over time to mimic as closely as possible the natural concentration curve in a healthy subject.

Mixing of the ingredients of a powder mixture before metering and forming doses may be done in all possible permutations, e.g. if more than one peptide is used, the peptides may be mixed with each other first and then added to a mixture of excipients, if necessary, but any permutation of the mixing steps may be used. The properties of the final powder mixture are decisive for the choice of mixing method, such that e.g. peptide stability is maintained, risk of particle segregation is eliminated and dose to dose relative standard deviation (RSD) is kept within specified limits, usually within 10% and preferably within 5%.

It is a further objective of the present invention to deliver a fine particle dose (FPD) of the at least one pure peptide powder, where the delivered FPD amounts to at least 40% by mass and typically 50-70% or more by mass of the active ingredients of the metered dose. In another aspect of the invention the at least one excipient of the metered dose is in a formulation where the MMAD of the particles is 10 μm or more, such that the at least one excipient acts as a carrier for the finely divided particles of the active peptide(s), besides diluting the potency of active ingredients and contributing to acceptable metering and dose forming properties of the powder mixture. When the metered dose is delivered to a user by means of a dry powder inhaler device (DPI), almost all of the excipient particle mass is deposited in the mouth and upper airways, because the aerodynamic diameters of excipient particles are generally too big to follow the inspiration air into the lung. Therefore, excipients are selected inter alia with a view to being harmless when deposited in these areas.

Suitable excipients for inclusion in a peptide formulation are to be found among the groups of monosaccarides, disaccarides, polylactides, oligo- and polysaccarides, polyalcohols, polymers, salts or mixtures from these groups, e.g. glucose, arabinose, lactose, lactose monohydrate, lactose anhydrous [i.e., no crystalline water present in lactose molecule], saccharose, maltose, dextrane, sorbitol, mannitol, xylitol, sodium chloride, calcium carbonate. A particular excipient is lactose.

In our experience many dry powder peptides are sensitive to moisture, which is also true of insulin. Thus, the moisture properties of any proposed excipient must be checked before it is chosen to be included in a formulation comprising a peptide, particularly insulin, regardless of the intended function of the proposed excipient. If an excipient gives off much water, after dose forming, it will negatively affect the active peptide in the dose, such that the FPD deteriorates rapidly after dose forming. Therefore, excipients to be mixed with peptides, particularly insulin, are to be selected among acceptable excipients, which have good moisture properties in the sense that the excipient will not adversely affect the FPD of the active peptide or peptides for the shelf life of the product, regardless of normal changes in ambient conditions during transportation and storage. Suitable “dry” excipients are to be found in the above-mentioned groups. In a particular embodiment of an insulin dose, lactose is selected as the preferred dry excipient and preferably lactose monohydrate. A reason for selecting lactose as excipient, is its inherent property of having a low and constant water sorption isotherm. Excipients having a similar or lower sorption isotherm can also be considered for use, provided other required qualities are met.

The dose size depends on the disorder and the selected peptide agent for adequate therapy, but naturally age, weight, gender and severity of the medical condition of the subject undergoing therapy are important factors. According to the present invention, a balanced, delivered fine particle dose (FPD) of pure peptide administered by inhalation generally spans a range from 10 μg to 50 mg, depending on substance. A physician of course normally prescribes a proper dose size. Depending on the potency of the active substance, such as human insulin, the active dose mass is optionally diluted to suit a particular method of dose forming. Further, the correct metered dose loaded into an inhaler to be used for pulmonary delivery must be adjusted for predicted losses such as retention and more or less efficient de-aggregation of the inhaled dose. A practical lower limit for volumetric dose forming is in a range 0.5 to 1 mg. Smaller doses are very difficult to produce and still maintain a low relative standard deviation between doses in the order of 10%. Typically, though, dose masses for inhalation are in a range from 0.1 to 50 mg and preferably in a range from 0.5 to 25 mg. A most suitable total dose mass in each particular case depends on the type of formulation selected for a certain poly-peptide drug, considering demands on the formulation set up from the point of view of, inter alia, medicament potency and dose metering and filling objectives. Further, requirements on the dose depending on the actual inhaler and the need, as already pointed out, to minimize powder retention in the device and maximize the delivered fine particle dose to a user, must also be considered when total dose mass is to be determined. Still further, careful consideration must be paid to what excipient or excipients are best qualified to be included in the formulation and what particle sizes should be present. For instance, large excipient particles (>25 μm) act as carriers of the micronized poly-peptide powder, while a small amount of small excipient particles (<10 μm) may improve the FPD of the poly-peptide dose.

Ambient conditions during dose forming, metering and container sealing should be closely controlled. The ambient temperature is preferably limited to 25° C. maximum and relative humidity preferably limited to 15% Rh maximum, but the actual permissible relative humidity depends on the specific formulation and some cases may require much less than 15%, even less than 5%. The powder formulation is also to be kept as dry as possible during the dose forming process. As already mentioned in the foregoing it is very important to control the electric properties of the powder and the use of charging and discharging, regardless of which method of dose forming is to be used. Fine powders pick up static electric charges extremely easily, which can be advantageously used in dose forming, if the charging and discharging is under proper control. Keeping the relative humidity low ensures that only a very small, acceptable amount of water is enclosed in the dose container together with the dose and not enough to present a threat to the stability of the moisture sensitive substance and the FPD of the peptide dose. The original fine particle fraction (FPF) of the medicament dose manifested in a high fine particle dose (FPD) of the metered dose of the active peptide powder at the packaging stage is thereby preserved in a dry, high barrier seal container enclosing the metered dose. Thus, when the metered dose is later delivered by a DPI it is unaffected for the shelf life of the medical product by normal variations in ambient conditions during handling, storage and delivery.

“High barrier seal” means a dry packaging construction or material or combinations of materials. A high barrier seal constitutes a high barrier against moisture diffusion and further implies that the seal itself is ‘dry’, i.e. it cannot give off measurable amounts of water to the load of powder it is protecting. A high barrier seal may for instance be made up of one or more layers of materials, i.e. technical polymers, aluminum or other metals, glass, silicon oxides etc that together constitutes the high barrier seal. If the high barrier seal is a foil, a 50 μm PCTFE/PVC pharmaceutical foil is the minimum required high barrier foil if a two week in-use stability for a moisture sensitive medicament shall be achieved. For longer in-use stabilities metal foils like aluminum foils from Alcan Singen can be used.

A “high barrier seal container” is a mechanical construction made to harbor and enclose a moisture sensitive dose of e.g. insulin. The high barrier container is built using high barrier seals constituting the enclosing, i.e. walls of the container. A high barrier seal container can be made in many different shapes, e.g. completely or partly spherical, cylindrical, box like etc. However, the volume of the container is preferably not bigger than necessary for loading and enclosing a metered dose, thereby minimizing the amount of moisture enclosed in the atmosphere. Another requirement is that the container is designed to facilitate opening thereof, preferably in a way that makes the enclosed dose accessible for direct aerosolization and entrainment of the powder in inspiration air during an inhalation. The time the dose is exposed to ambient air is thereby minimized.

A high barrier seal container to be loaded with a dose of a peptide medicament is preferably made from aluminum foils of high barrier seal quality and approved to be in direct contact with pharmaceutical products. Aluminum foils that work properly in these aspects generally contain technical polymers laminated with aluminum foil to give the foil the correct mechanical properties to avoid cracking of the aluminum during forming. Sealing of the formed containers is normally performed by using a thinner cover foil of pure aluminum or laminated aluminum and polymer. The container and cover foils are then sealed together using at least one of several possible methods, for instance:
using a heat sealing lacquer, through pressure and heat;
using heat and pressure to fuse the materials together;
ultrasonic welding of the materials in contact.

The sealed, dry, high barrier container of the present invention that is directly loaded with a peptide dose may be in the form of a blister and it may e.g. comprise a flat dose bed or a formed cavity in aluminum foil or a molded cavity in a polymer material, using a high barrier seal foil against ingress of moisture, e.g. of aluminum or a combination of aluminum and polymer materials. The sealed, dry, high barrier container may form a part of an inhaler device or it may form a part of a separate item intended for insertion into an inhaler device for administration of pre-metered doses. A particular embodiment of a sealed high barrier container used in an adapted DPI has the following data:
Container internal volume: 100 mm³
Effective diffusion area: 46 mm²
Diffusion constant: 0.044 g/m² for 24 hours at 23° C. and differential Rh=50%

Expressed in a different way, the diffusion of water into the container was in this case at a rate of 20 g/m³ per 24 hours at 23° C. and at a presumed driving difference in Rh of 50%. Tests have shown that the container in the example was adequate for protecting a dose of a particularly moisture sensitive substance for 14 days. Thus, the present invention teaches that e.g. a sealed high barrier container of the size above holding a dose of the substance should not have a water transmission rate of more than 20 g/m³ for 24 hours at 23° C. and differential Rh=50%, to be suitable for an in-use time of maximum 2 weeks. The results from the tests may be transposed into a set of demands put on a different type of container, e.g. a blister. A blister of similar size to the container in the example would have to be made using a typical high quality material like 50 μm PCTFE/PVC, which just meets the diffusion constant of the container in the example (=0.118 g/m² when re-calculated to at 38° C. and 90% Rh). If a container loaded with a dose of the substance is intended to be in use for longer periods than 2 weeks, then a more moisture tight container must be used to protect the FPD.

In a further aspect of the present invention a medical product is disclosed comprising a metered dose of at least one, finely divided, dry powder of a pure, peptide medicament optionally in a mixture with at least one biologically acceptable excipient loaded and sealed into a high barrier seal container. The container is thus protecting the dose from ingress of moisture and other foreign matter, thereby preserving the FPD of the peptide medicament. Deterioration of the FPD is further protected by enclosing as little moisture as possible inside the container together with the dose by keeping the humidity in the atmosphere during dose metering and forming to a sufficiently low level, and optionally by choosing the biologically acceptable excipient with as low sorption coefficient as possible. For instance, the humidity in the atmosphere where the powder is handled immediately prior to metering and forming should be kept below 15% Rh and preferably below 10% Rh, more preferably below 5% Rh and most preferably below 1% Rh. The disclosed medical product warrants that the quality of the delivered dose is high and intact over the full shelf life period and the in-use period of the product.

In a particular embodiment of the medical product, at least one recombinant, human insulin is selected as the peptide medicament.

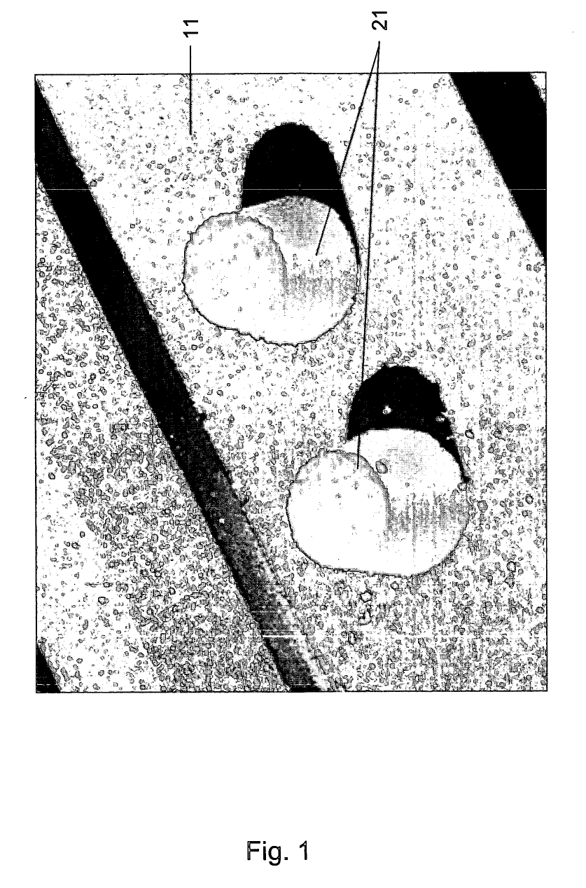

In FIGS. 1, 2, 3 and 4 reference numbers 11-32 indicate like elements throughout the different embodiments of doses of a dry powder medicament comprising a peptide powder formulation loaded onto a dose bed of a container as illustrated and presented here as non-limiting examples.

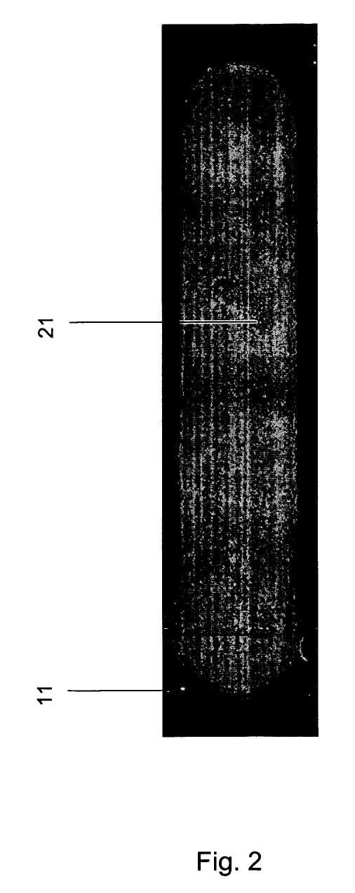

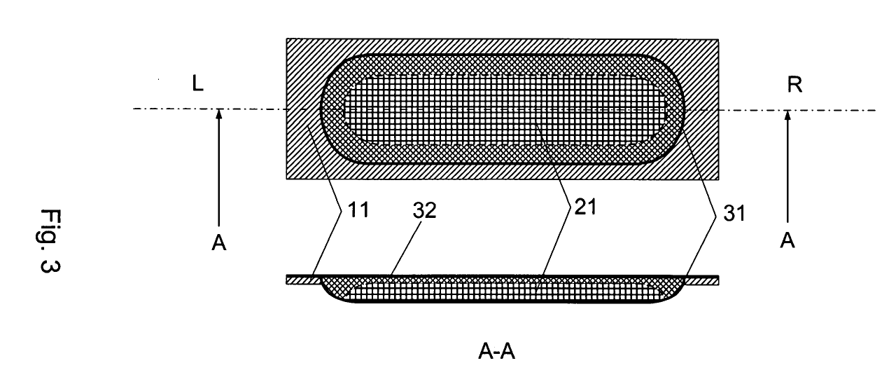

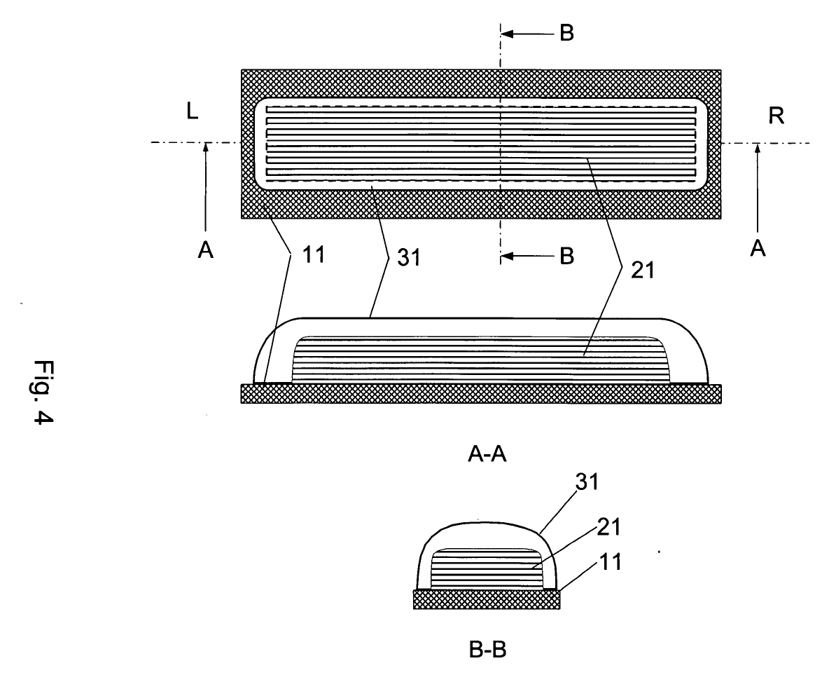

In FIG. 1 a close-up illustration is shown of two metered volumetrically formed doses 21 loaded onto a common dose bed 11. FIG. 2 illustrates a close-up view of a metered electro-dynamically formed dose 21 onto an oblong dose bed 11. FIG. 3 illustrates a side and a top view of a dose 21 loaded onto a dose bed 11 of a high barrier container, the dose sealed moisture-tight by a high barrier seal 31. FIG. 4 illustrates two side views and a top view of another embodiment of a dose 21 loaded onto a dose bed 11 of a high barrier container, the dose sealed moisture tight by a high barrier seal 31 and 32.

As used herein, the phrases “selected from the group consisting of,” “chosen from,” and the like include mixtures of the specified materials.

All references, patents, applications, tests, standards, documents, publications, brochures, texts, articles, instructions, etc. mentioned herein are incorporated herein by reference. Where a numerical limit or range is stated, the endpoints are included. Also, all values and sub-ranges within a numerical limit or range are specifically included as if explicitly written out.

In the context of this document all references to ratios, including ratios given as percentage numbers, are related to mass, if not explicitly said to be otherwise.

What is claimed:

1. A metered medication dose of a peptide medicament in dry powder form, to be made available in an adapted dry powder inhaler, wherein
said peptide medicament comprises at least one micronized peptide powder and optionally at least one biologically acceptable excipient in dry powder form acting only as a carrier or diluent for the peptide(s);
the at least one peptide powder is in a pure form, without any substances included in the dose that are intended to change or enhance one, some or all properties of the at least one peptide;
the metered medication dose is adapted for a prolonged dose delivery directly from a high barrier seal container enclosing the dose;
the metered medication dose is arranged to be aerosolized gradually into inspiration air by the suction effort made by a user of the inhaler, whereby more than 40% by mass of the at least one peptide powder, contained in the metered dose, leaves the inhaler as a fine particle dose.

2. The metered medication dose according to claim 1, wherein
a total mass of peptide(s) powder in the metered dose is in a range from 10 μg to 50 mg of a total dose mass in a range from 0.1 mg to 50 mg, more preferably in a range from 0.5 to 25 mg.

3. The metered medication dose according to claim 1, wherein
said at least one peptide powder is selected from a group comprising rapid, intermediate and slow acting insulin including insulin analogues, C-peptide of insulin, alpha1-proteinase inhibitor, glucagons, glucagon-like peptides, dipeptidyl-peptidase-4, interleukin 1, parathyroid hormone, genotropin, colony stimulating factors, erythropoietin, interferons, calcitonin, factor VIII, alpha-1-antitrypsin, follicle stimulating hormones, LHRH agonist and IGF-1.

4. The metered medication dose according to claim 1, wherein
said at least one peptide powder is recombinant, human insulin powder;
a mass of the insulin powder in the metered dose is in a range from 400 μg to 20 mg.

5. The metered medication dose according to claim 1, wherein
said at least one peptide powder is a glucagon or a glucagon-like peptide-1 powder.

6. The metered medication dose according to claim 1, wherein
the at least one peptide powder is dipeptidyl-peptidase-4 powder.

7. The metered medication dose according to claim 1, wherein
the at least one peptide powder is parathyroid hormone powder.

8. The metered medication dose according to claim 1, wherein
the prolonged dose delivery is not shorter than 0.1 s and not longer than 5 s, but preferably in a range from 0.2 s to 2 s.

9. The metered medication dose according to claim 1, wherein
the suction effort produces at least 2 kPa of suction energy.

10. The metered medication dose according to claim 1, wherein
more than 50% by mass, preferably more than 60% and most preferably more than 70% of the at least one peptide powder, contained in the metered dose, leaves the inhaler as a fine particle dose (FPD).

11. The metered medication dose according to claim 1, wherein
said at least one peptide powder is presented having a mass median aerodynamic diameter in a range from 1 to 5 μm and at least 90% of its mass in this range.

12. The metered medication dose according to claim 1, wherein
said at least one, optional dry excipient comprises particles having a diameter of 25 μm or more, and
said at least one, optional dry excipient comprises an excipient selected from a group consisting of monosaccarides, disaccarides, polylactides, oligo- and polysaccarides, polyalcohols, polymers, salts or mixtures thereof.

13. The metered medication dose according to claim 1, wherein
said peptide medicament consists of the at least one micronized peptide powder and optionally the at least one biologically acceptable excipient in dry powder form acting only as a carrier or diluent for the peptide(s).

14. A medical product comprising a metered dose of a dry powder peptide medicament loaded in a sealed container, made to fit into an adapted dry powder inhaler, wherein
said peptide medicament comprises at least one micronized peptide powder and optionally at least one biologically acceptable excipient powder in dry powder form acting only as a carrier or diluent for the peptide(s);
the at least one peptide powder is in a pure form, without any substances included in the dose that are intended to change or enhance one, some or all properties of the peptide;
the sealed container enclosing the metered dose is a dry, moisture-tight, high barrier seal container adapted for a prolonged dose delivery from the container using the adapted dry powder inhaler;
the metered dose is arranged to be aerosolized gradually into inspiration air directly from the container, when opened, by the suction effort made by a user of the adapted inhaler, whereby more than 40% by mass of the at least one peptide powder, contained in the metered dose, leaves the inhaler as a fine particle dose;

15. The medical product according to claim 14, wherein
said at least one peptide powder is recombinant, human insulin powder;
a mass of the insulin powder in the metered dose is in a range from 400 μg to 20 mg.

16. The medical product according to claim 14, wherein
said at least one peptide powder is a glucagon or a glucagon-like peptide-1 powder.

17. The medical product according to claim 14, wherein
said at least one peptide powder is dipeptidyl-peptidase-4 powder.

18. The medical product according to claim 14, wherein
said at least one peptide powder is parathyroid hormone powder.

19. The medical product according to claim 14, wherein
the prolonged dose delivery is not shorter than 0.1 s and not longer than 5 s, but preferably in a range from 0.2 s to 2 s.

20. The medical product according to claim 14, wherein
the suction effort by the user produces at least 2 kPa of suction energy.

21. The medical product according to claim 14, wherein
more than 50% by mass, preferably more than 60% and most preferably more than 70% of the at least one peptide powder, contained in the metered dose, leaves the inhaler as a fine particle dose (FPD).

22. The medical product according to claim 14, wherein
a total mass of peptide(s) powder in the metered dose is in a range from 10 μg to 50 mg of a total dose mass in a range from 0.1 mg to 50 mg, preferably in a range from 0.5 to 25 mg.

23. The medical product according to claim 14, wherein
said at least one peptide powder is presented having a mass median aerodynamic diameter in a range from 1 to 5 μm and at least 90% of its mass in this range.

24. The medical product according to claim 14, wherein
said at least one, optional dry excipient comprises particles having a diameter of 25 μm or more, and
the at least one, optional dry excipient comprises an excipient selected from a group consisting of monosaccarides, disaccarides, polylactides, oligo- and polysaccarides, polyalcohols, polymers, salts or mixtures thereof.

25. The medical product according to claim 14, wherein
the dry, high barrier seal comprises a material selected from the group consisting of metals, thermoplastics, glass, silicon, silicon oxides, and combinations thereof.

26. The medical product according to claim 14, wherein
the dry powder medicament dose in the container is formed using either volumetric, gravimetric or electric field dose forming methods, or combinations thereof.

27. The medical product according to claim 14, wherein
the dry, high barrier seal comprises a formed or flat aluminum foil, optionally laminated with at least one polymer.

28. The medical product according to claim 14, wherein
said container forms a cavity molded from an aluminum foil optionally laminated with at least one polymer providing high barrier seal properties.

29. The medical product according to claim 14, wherein
said container is a part of a dry powder inhaler.

30. The medical product according to claim 14, wherein
said container is a separate part adapted for insertion into a dry powder inhaler.

31. The medical product according to claim 14, wherein
said container is a separate part comprising a primary part adapted for insertion into a dry powder inhaler and a secondary part enclosing the primary part in a moisture-tight package.

32. The medical product according to claim 14, wherein
said at least one peptide powder is selected from a group comprising rapid, intermediate and slow acting insulin including insulin analogues, C-peptide of insulin, alpha1-proteinase inhibitor, glucagons, glucagon-like peptides, dipeptidyl-peptidase-4, parathyroid hormone, interleukin 1, parathyroid hormone, genotropin, colony stimulating factors, erythropoietin, interferons, calcitonin, factor VIII, alpha-1-antitrypsin, follicle stimulating hormones, LHRH agonist and IGF-1.

33. The medical product according to claim 14, wherein
said peptide medicament consists of at least one peptide powder is selected from a group comprising rapid, intermediate and slow acting insulin including insulin analogues, C-peptide of insulin, alpha1-proteinase inhibitor, glucagons, glucagon-like peptides, dipeptidyl-peptidase-4, parathyroid hormone, interleukin 1, parathyroid hormone, genotropin, colony stimulating factors, erythropoietin, interferons, calcitonin, factor VIII, alpha-1-antitrypsin, follicle stimulating hormones, LHRH agonist and IGF-1.

34. The medical product according to claim 14, wherein
said peptide medicament comprises at least a first and a second micronized peptide powder, the first and the second one selected from a group comprising rapid, intermediate and slow acting insulin including insulin analogues, C-peptide of insulin, glucagons, glucagon-like peptides and dipeptidyl-peptidase-4.

35. The metered medication dose according to claim 1, wherein
said peptide medicament comprises at least a first and a second micronized peptide powder, the first and the second one selected from a group comprising rapid, intermediate and slow acting insulin including insulin analogues, C-peptide of insulin, glucagons, glucagon-like peptides and dipeptidyl-peptidase-4.
